(19) United States
(12) Patent Application Publication
(10) Pub. No.: US 2019/0134303 A1
(43) Pub. Date: May 9, 2019

(54) Needleless IV Injection Port

(71) Applicant: Fresenius Kabi Deutschland GmbH, Bad Homburg (DE)

(72) Inventors: Dana Wm. Ryan, Mt. Juliet, TN (US); Anthony E. Ryherd, Austin, TX (US); James M. Kaiser, Austin, TX (US)

(21) Appl. No.: 16/026,988

(22) Filed: Jul. 3, 2018

Related U.S. Application Data
(63) Continuation of application No. 15/629,000, filed on Jun. 21, 2017, now abandoned.

(51) Int. Cl. A61M 5/168; A61M 5/162

(52) U.S. Cl. CPC A61M 5/168; A61M 2039/262; A61M 5/162

(57) ABSTRACT

An injection port assembly comprises a body having a first mating structure and a second mating structure coupled together. A resilient barrier is received within the body and compressible between a less compressed first position in which fluid flow through the injection port assembly is blocked, to a more compressed second position in which fluid flow through the injection port assembly is permitted. A hollow cannula is coupled to the first mating structure and configured to be received within the resilient barrier. The hollow cannula has a distal end portion configured to extend through the resilient barrier when the resilient barrier is in the more compressed second position. The hollow cannula is opaque and both the second mating structure and the resilient barrier are transparent, so that any fluid leakage into the internal cavity of the resilient barrier is visible through the resilient barrier and the second mating structure against an opaque background provided by the opaque cannula.

### BACKGROUND OF THE INVENTION

### 1. Field of the Invention

The present disclosure relates generally to medical intravenous administration line connectors. More particularly, this disclosure pertains to a needless, intermittent, injection port for safe infusion of IV fluids, antibiotics, lipids, blood, blood components or drug products and/or blood aspiration in intravenous and blood administration therapy.

### 2. Description of the Prior Art

One important aspect of needless injection ports is the ability of the medical worker using the injection port to visualize the fluids flowing through the injection port. This is important for several reasons. First, during the primary usage of the injection port it may be important to visualize the proper flow of fluids through the injection port. After the primary usage, it may be important to visualize the passages through the injection port during the flushing of the injection port. Finally, during all usages of the injection port it is important to be able to detect any malfunction of the injection port, such as leakage between its various components.

These issues have led to the development of so-called “clear” injection ports, wherein all of the components of the injection port are transparent, thus allowing visualization of the fluids in the injection port.

There is a continuing need for improvements in such injection ports to make the same more reliable in use, and to make malfunctions more readily detectable.

### SUMMARY OF THE INVENTION

In one aspect, an injection port assembly may include a body having first and second mating structures configured to be coupled together. A resilient barrier is configured to be received within the body and is compressible from a less compressed first position in which fluid flow through the injection port assembly is blocked, to a more compressed second position in which fluid flow through the injection port assembly is permitted. The resilient barrier includes an internal cavity. A hollow cannula is coupled to the first mating structure and is configured to be received within the resilient barrier. The hollow cannula has a cannula distal end portion configured to extend through the resilient barrier when the resilient barrier is in the more compressed second position. The cannula distal end portion may have at least one outlet window. At least a portion of the hollow cannula may be opaque, and at least a portion of both the second mating structure and the resilient barrier may be transparent, so that any fluid leakage into the internal cavity of the resilient barrier is visible through the resilient barrier and the second mating structure against an opaque background provided by the opaque cannula.

The entire cannula may be opaque, and the entire resilient barrier and second mating structure may be transparent.

In one aspect, the injection port assembly may be particularly adapted for visualization of blood leakage, and the opaque cannula may have a color that is lighter than blood so as to provide a contrasting background to any blood leakage outside of the cannula.

The opaque cannula may have a color other than red, so as to contrast to any blood leakage outside of the cannula.

The opaque cannula may have a pastel color so as to contrast with blood in the internal cavity of the resilient barrier.

In one embodiment, the opaque cannula may be colored a light pastel blue.

The opaque cannula may be integrally formed with the first mating structure and the entire first mating structure may be opaque.

The hollow cannula has an internal fluid passageway, and the cannula is sufficiently opaque that blood inside the internal fluid passageway is not visible through the cannula.

In another embodiment an injection port assembly may include a body having a first mating structure and a second mating structure configured to be coupled to the first mating structure. A resilient barrier may be configured to be received within the body and compressible from a less compressed first position in which fluid flow through the injection port assembly is blocked, to the more compressed second position in which fluid flow through the injection port assembly is permitted. The resilient barrier may include an internal cavity. A hollow cannula may be coupled to the first mating structure and configured to be received within the internal cavity of the resilient barrier. The hollow cannula may have a cannula end portion configured to extend through the resilient barrier when the resilient barrier is in the more compressed second position. The cannula may have an internal fluid passageway. At least a portion of the hollow cannula is opaque so that the internal fluid passageway of the cannula is not visible through the cannula. At least a portion of both the second mating structure and the resilient barrier may be transparent so that the internal cavity of the resilient barrier is visible through the resilient barrier and the second mating structure.

Numerous objects, features and advantages of the present invention will really be readily apparent to those skilled in the art upon reading of the following disclosure when taken in conjunction with the accompanying drawings.

### BRIEF DESCRIPTION OF THE DRAWINGS

FIG. 1 is an exploded view of the components of the injection port assembly.

FIG. 2 is an assembled elevation view of the injection port assembly of FIG. 1.

FIG. 3 is an elevation cross-section view of the injection port assembly of FIGS. 1 and 2 taken along line 3-3 of FIG. 2.

FIG. 4 is an elevation cross-section view of the injection port assembly of FIG. 1 in the less compressed position of the resilient barrier. A syringe is shown in position about to be engaged with the injection port assembly.

FIG. 5 is a view similar to FIG. 4, the syringe having been engaged with the injection port assembly to move the resilient barrier to its more compressed second position in which fluid flow through the injection port assembly is permitted.

FIG. 6 is an exploded view of the components of an alternative embodiment of the injection port assembly.

FIG. 7 is an assembled elevation cross-section view of the injection port assembly of FIG. 6.

### DETAILED DESCRIPTION

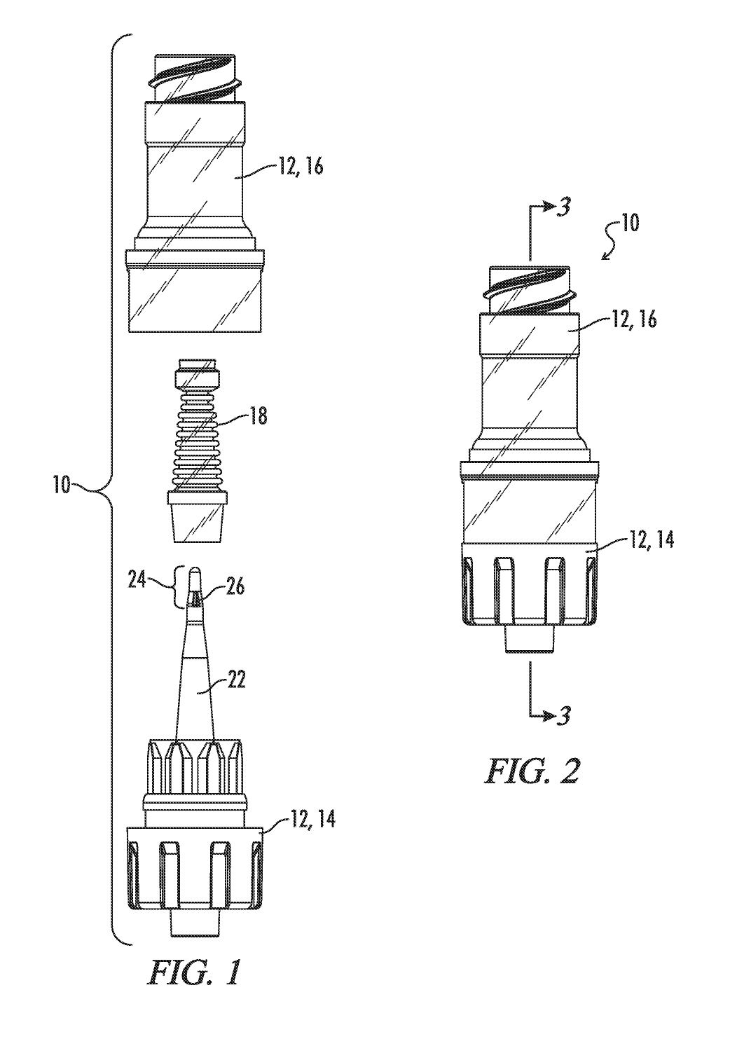

Referring now to the drawings, and particularly to FIGS. 1 and 2, an injection port assembly is shown and generally designated by the number 10. The injection port assembly 10 includes a body 12 having a first mating structure 14 and a second mating structure 16 configured to be coupled to the first mating structure 14.

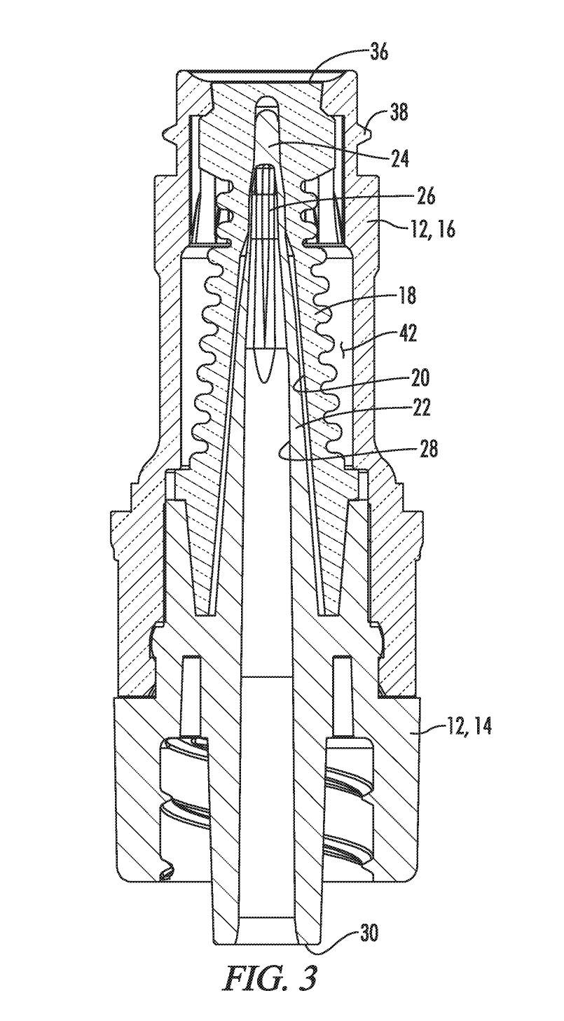

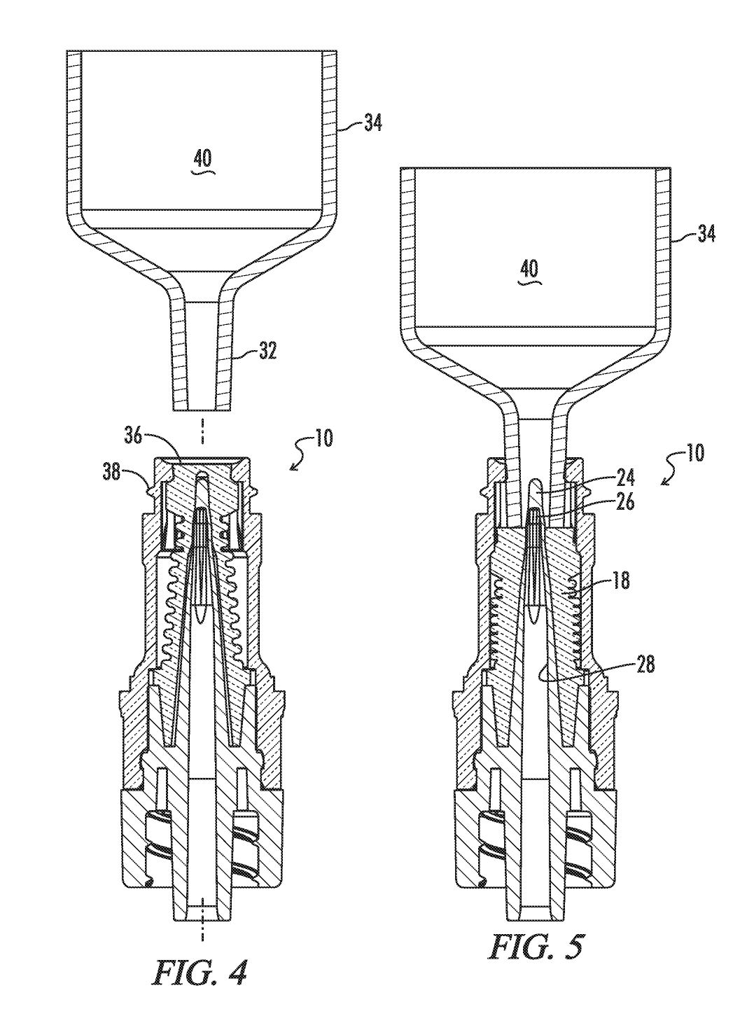

A resilient barrier 18 is configured to be received within the body 12 and is compressible from a less compressed first position as seen in FIG. 4, in which fluid flow through the injection port assembly 10 is blocked, to a more compressed second position as seen in FIG. 5, in which fluid flow through the injection port assembly 10 is permitted. The resilient barrier 18 has an internal cavity 20, as seen in FIG. 3.

A hollow cannula 22 is coupled to the first mating structure 14 and configured to be received within the resilient barrier 18. The hollow cannula 22 has a cannula distal end portion 24 configured to extend through the resilient barrier 18 when the resilient barrier 18 is in the more compressed second position as seen in FIG. 5. The cannula distal end portion 24 has at least one outlet window 26 defined therein.

The hollow cannula 22 is preferably integrally formed with the first mating structure 14. Hollow cannula 22 has an internal fluid passageway 28 defined therein, communicating a proximal end 30 of the first mating structure 14 with the outlet windows 26.

As illustrated in FIGS. 4 and 5, the connector 10 is constructed to be engaged by a male Luer tip 32 of a syringe 34 or other medical component. The male Luer tip 32 engages the distal end 36 of resilient barrier 18 and forces the same downward over the hollow cannula 22 so that the distal portion 24 thereof is exposed, allowing fluid communication between the syringe 34 and the windows 26 of the internal fluid passageway 28 of cannula 22.

The syringe 34 may also include a Luer lock connection (not shown) having female threads which engage the male threads 38 of the second mating structure 16.

As will be appreciated in viewing FIG. 5, when the syringe 34 is engaged with the injection port assembly 10 and moves the resilient barrier 18 to its more compressed second position, fluid communication is provided between the interior 40 of the syringe 34 and the internal fluid passageway 28 of the hollow cannula 22. This allows fluid to either flow out of the syringe 34 and through the internal fluid passageway 28 to other components of the intravenous assembly to move fluids into a patient's body, or fluids such as blood may be drawn from the patient's body upward through the internal fluid passageway 28 into the interior 40 of the syringe 34. When fluid flows in either direction a properly functioning injection port assembly 10 will not have any fluid leakage outside of the fluid pathway just described.

In the event of undesired leakage, it is possible that fluids may find their way into the internal cavity 20 of the resilient barrier 18, or into an annulus 42 between the resilient barrier 18 and the second mating structure for 16. The present invention is particularly adapted for the ease of visualization and detection of such leakage into either the internal cavity 20 of resilient barrier 18 or into the annulus 42 between the resilient barrier 18 and the second mating structure 16. This is accomplished by making at least a portion of the hollow cannula 22, and preferably the entire cannula 22 and the entire first mating structure 14, from an opaque material so that the internal fluid passageway 28 is not visible through the opaque cannula 22. Particularly fluids such as blood contained in the internal fluid passageway 28, should not be visible through the opaque cannula 22. Furthermore, at least a portion of the resilient barrier 18 and at least a portion of the second mating structure 16 of body 12 are made from transparent materials so that the internal cavity 20 of the resilient barrier 18 and the exterior surface of cannula 22 are readily visible through the second mating structure 16 and the resilient barrier 18. Preferably the entire resilient barrier 18 and the entire second mating structure 16 are made from transparent materials.

Thus any fluid leakage into the internal cavity 20 of resilient barrier 18 is readily visible through the resilient barrier 18 and the second mating structure 16 against the opaque background provided by the exterior surface of the opaque cannula 22.

This is contrasted to the situation in the so called “clear” injection ports of the prior art in which all components including the cannula are clear. If the cannula is also clear, then for example when withdrawing blood from a patient there is always blood visible in the internal fluid passageway 28 of the cannula 22 and thus it can be difficult to visualize blood leakage into the internal cavity 20 of the resilient barrier 18.

Depending upon the fluids which are expected to be transmitted through the injection port assembly 10, the color of the opaque cannula 22 may be selected so as to provide the greatest contrast for ease of visualization of any fluids which might leak into the internal cavity 20 of resilient barrier 18 or into the annulus 42.

For example, when the injection port assembly 10 is anticipated to be used for drawing blood samples from the patient, and when it is desired to be able to visualize any blood leakage, the color of the opaque cannula 22 may be selected so as to provide a distinct contrast with any blood which leaks into the internal cavity 20. For example, the opaque cannula 22 may have a color which is lighter than blood. Preferably in this situation the opaque cannula 22 will have a color other than red. The opaque cannula 22 may have a pastel color such as to contrast with blood in the internal cavity 20. The cannula 22 in a preferred embodiment may be colored a light pastel blue, which provides a distinct contrast with any blood leakage into the internal cavity 20. A white colored opaque cannula 22 also provides a good contrasting background to blood.

One particularly suitable light pastel blue color is Pantone PMS-544. Another suitable light pastel blue color is Pantone PQ-7541C. Other suitable opaque pastel background colors, depending upon the fluid desired to be visualized, may include:
Pantone 11-0603 Pastel Parchment;
Pantone 11-0616 Pastel Yellow
Pantone 12-1007 Pastel Rose Tan
Pantone 12-4607 Pastel Blue
Pantone 13-0116 Pastel Green
Pantone 13-6309 Pastel Turquoise.

If the injection port assembly is to be optimized for visualization of specific fluids to be injected into the patient's body, then a suitable contrasting opaque color for the cannula 22 may be selected.

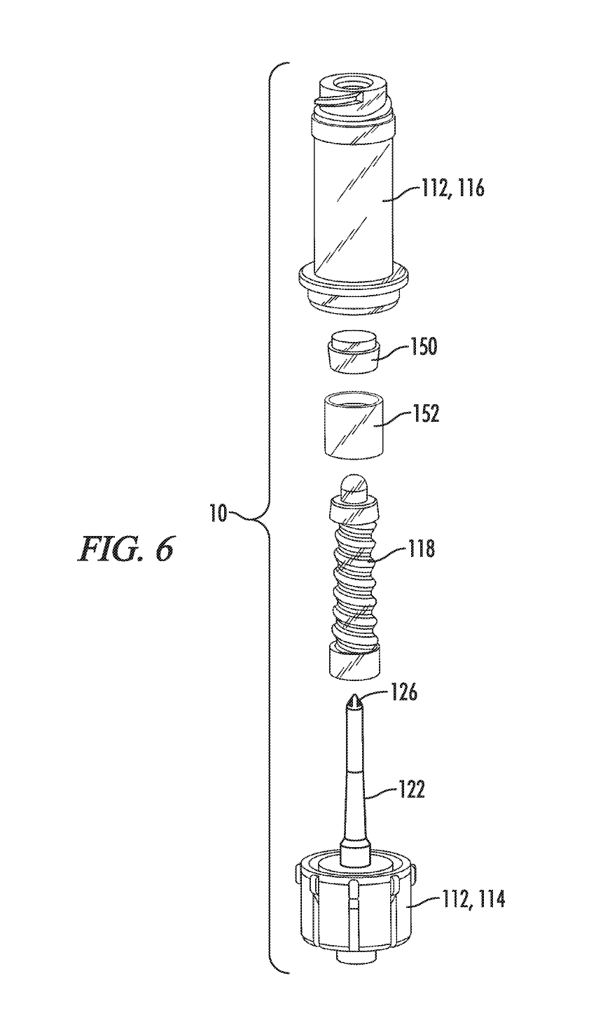

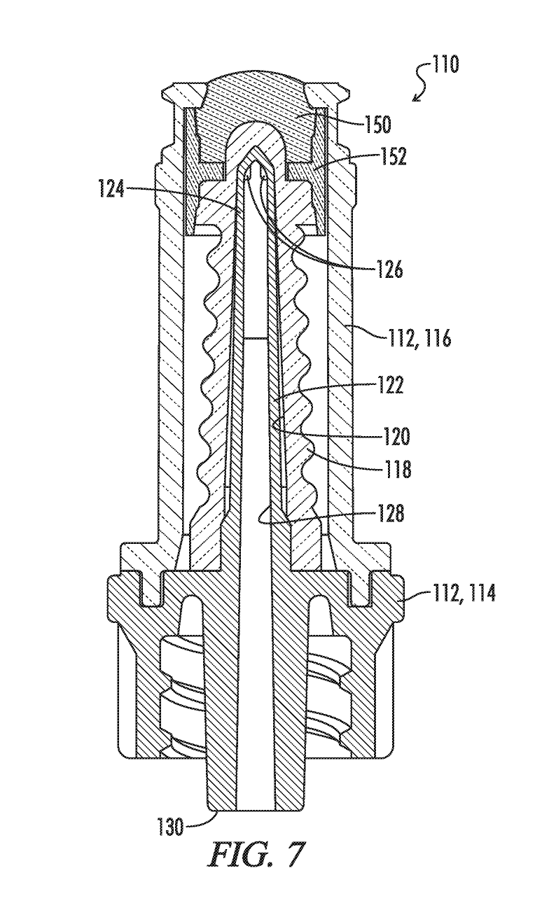

### The Embodiment of FIGS. 6 and 7:

FIGS. 6 and 7 show an alternative embodiment of an injection port assembly incorporating the present invention, which is generally designated by the number 110. The general construction of the components of the injection port assembly 110 and the operation thereof, but not the transparency or opaqueness of the materials from which it is constructed, are generally in accordance with the teachings of U.S. Pat. No. 8,096,525 to Ryan, the details of which are incorporated herein by reference.

The injection port assembly 110 includes a body 112 having a first mating structure 114 and a second mating structure 116 configured to be coupled to the first mating structure 114.

A resilient barrier 118 is configured to be received within the body 112 and is compressible from a less compressed first position in which fluid flow through the injection port assembly 110 is blocked, to a more compressed second position in which fluid flow through the injection port assembly 110 is permitted. The resilient barrier 118 has an internal cavity 120.

A hollow cannula 122 is coupled to the first mating structure 114 and configured to be received within the resilient barrier 118. The hollow cannula 122 has a cannula distal end portion 124 configured to extend through the resilient barrier 118 when the resilient barrier 118 is in the more compressed second position. The cannula distal end portion 124 has at least one outlet window 126 defined therein.

The hollow cannula 122 is preferably integrally formed with the first mating structure 114. Hollow cannula 122 has an internal fluid passageway 128 defined therein, communicating a proximal end 130 of the first mating structure 114 with the outlet windows 126.

At least a portion of the hollow cannula 122, and preferably the entire hollow cannula 122 and the entire first mating structure 114, are constructed from an opaque material so that the internal fluid passageway 128 is not visible through the opaque cannula 122. Furthermore, at least a portion of the resilient barrier 118 and at least a portion of the second mating structure 116 of body 112 are made from transparent materials so that the internal cavity 120 of the resilient barrier 118 and the exterior surface of cannula 122 are readily visible through the second mating structure 116 and the resilient barrier 118. Preferably the entire second mating structure 116 and the entire resilient barrier 118 are made from transparent material.

The injection port assembly 110 also includes a separate septum 150 and an H-guide 152 which are assembled with the upper end of the resilient barrier 118 as generally seen in FIG. 7. The H-guide has an “H” shape cross-section. The H-guide 152 is connected to the separate septum 150 and the resilient barrier 118 to guide the end of the resilient barrier 118 and the separate septum 150 in sliding motion within the second mating structure 116. Although it is preferable that the separate septum 150 and the H-guide 152 are also constructed of a clear material, it is not required. It will be appreciated that even if the separate septum 150 and/or the H-guide 152 are constructed of opaque material, at least a portion of the internal cavity 120 of the resilient barrier 118 will be visible through the resilient barrier 118 and the second mating structure 116.

Thus it is seen that the apparatus of the present invention readily achieves the ends and advantages mentioned as well as those inherent therein. While certain preferred embodiments of the invention have been illustrated and described for purposes of the present disclosure, numerous changes in the arrangement and construction of parts and steps may be made by those skilled in the art, which changes are encompassed within the scope and spirit of the present invention as defined by the appended claims.

What is claimed is:

1. An injection port assembly, comprising:
a body having a first mating structure and a second mating structure configured to be coupled to the first mating structure;
a resilient barrier configured to be received within the body and compressible from a less compressed first position in which fluid flow through the injection port assembly is blocked to a more compressed second position in which fluid flow through the injection port assembly is permitted, the resilient barrier including an internal cavity;
a hollow cannula coupled to the first mating structure and configured to be received within the resilient barrier, the hollow cannula having a cannula end portion configured to extend through the resilient barrier when the resilient barrier is in the more compressed second position, the cannula end portion having at least one outlet window;
wherein at least a portion of the hollow cannula is opaque and at least a portion of both the second mating structure and the resilient barrier are transparent.

2. The injection port assembly of claim 1, wherein:
fluid leakage into the internal cavity of the resilient barrier is visible though the resilient barrier and the second mating structure against an opaque background provided by the opaque cannula.

3. The injection port assembly of claim 1, wherein:
the opaque portion of the cannula has a color lighter than blood.

4. The injection port assembly of claim 1, wherein:
the opaque portion of the cannula has a color other than red.

5. The injection port assembly of claim 1, wherein:
the opaque portion of the cannula has a pastel color such as to contrast to blood in the internal cavity of the resilient barrier.

6. The injection port assembly of claim 1, wherein:
the opaque portion of the cannula is colored a light pastel blue.

7. The injection port assembly of claim 1, wherein:
the opaque portion of the cannula is integrally formed with the first mating structure and the entire first mating structure is opaque.

8. The injection port assembly of claim 1, wherein:
the hollow cannula has an internal fluid passageway, and blood inside the internal fluid passageway is not visible through the opaque portion of the cannula.

9. The injection port assembly of claim 1, wherein:
the hollow cannula has an internal fluid passageway and the internal fluid passageway is not visible through the opaque portion of the cannula.

10. The injection port assembly of claim 1, wherein:
at least a portion of the internal cavity of the resilient barrier is visible through the resilient barrier and the second mating structure.

11. The injection port assembly of claim 1, wherein:
the entire cannula is opaque and the entire second mating structure and the entire resilient barrier are transparent.

12. The injection port assembly of claim 1, further comprising:
a separate septum engaging an end of the resilient barrier;
a guide connected to the separate septum and the resilient barrier to guide the end of the resilient barrier and the separate septum in sliding motion within the second mating structure;
wherein the separate septum and the guide are both transparent.

13. An injection port assembly, comprising:
a body having a first mating structure and a second mating structure configured to be coupled to the first mating structure;
a resilient barrier configured to be received within the body and compressible from a less compressed first position in which fluid flow through the injection port assembly is blocked to a more compressed second position in which fluid flow through the injection port assembly is permitted, the resilient barrier including an internal cavity;
a hollow cannula coupled to the first mating structure and configured to be received within the internal cavity of the resilient barrier, the hollow cannula having a cannula end portion configured to extend through the resilient barrier when the resilient barrier is in the more compressed second position, the cannula having an internal fluid passageway;
wherein at least a portion of the hollow cannula is opaque so that the internal fluid passageway of the cannula is not visible through the opaque portion of the cannula; and
wherein at least a portion of both the second mating structure and the resilient barrier are transparent so that at least a portion of the internal cavity of the resilient barrier is visible though the resilient barrier and the second mating structure.

14. The injection port assembly of claim 13, wherein:
the opaque portion of the cannula has a color lighter than blood.

15. The injection port assembly of claim 13, wherein:
the opaque portion of the cannula has a color other than red.

16. The injection port assembly of claim 13, wherein:
the opaque portion of the cannula has a pastel color such as to contrast to any blood which leaks into the internal cavity of the resilient barrier.

17. The injection port assembly of claim 13, wherein:
the opaque portion of the cannula is colored a light pastel blue.

18. The injection port assembly of claim 13, wherein:
the opaque portion of the cannula is integrally formed with the first mating structure and the entire first mating structure is opaque.

19. The injection port assembly of claim 13, wherein:
any blood inside the internal fluid passageway of the hollow cannula is not visible through the opaque portion of the cannula.

20. The injection port assembly of claim 13, wherein:
the entire cannula is opaque and the entire second mating structure and the entire resilient barrier are transparent.

21. The injection port assembly of claim 13, further comprising:
a separate septum engaging an end of the resilient barrier;
a guide connected to the separate septum and the resilient barrier to guide the end of the resilient barrier and the separate septum in sliding motion within the second mating structure;
wherein the separate septum and the guide are both transparent.
